(19) United States
(12) Patent Application Publication
(10) Pub. No.: US 2008/0293991 A1
(43) Pub. Date: Nov. 27, 2008

(54) Oligomerization Using Molecular Sieve Catalysts

(76) Inventors: Jihad M. Dakka, Whitehouse Station, NJ (US); Hans K.T. Goris, Laakdal (BE)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 11/912,844

(22) PCT Filed: May 26, 2006
(86) PCT No.: PCT/EP2006/005074

§ 371 Date: Jun. 4, 2008

(30) Foreign Application Priority Data
May 31, 2005 (GB) 0510887.3

(51) Int. Cl. C07C 2/02; B01J 38/06

(52) U.S. Cl. 585/520; 502/55

(57) ABSTRACT

Treatment with steam, especially at sub-atmospheric pressure, rejuvenates deactivated zeolites that have been used as catalysts for liquid phase or dense phase oligomerization.

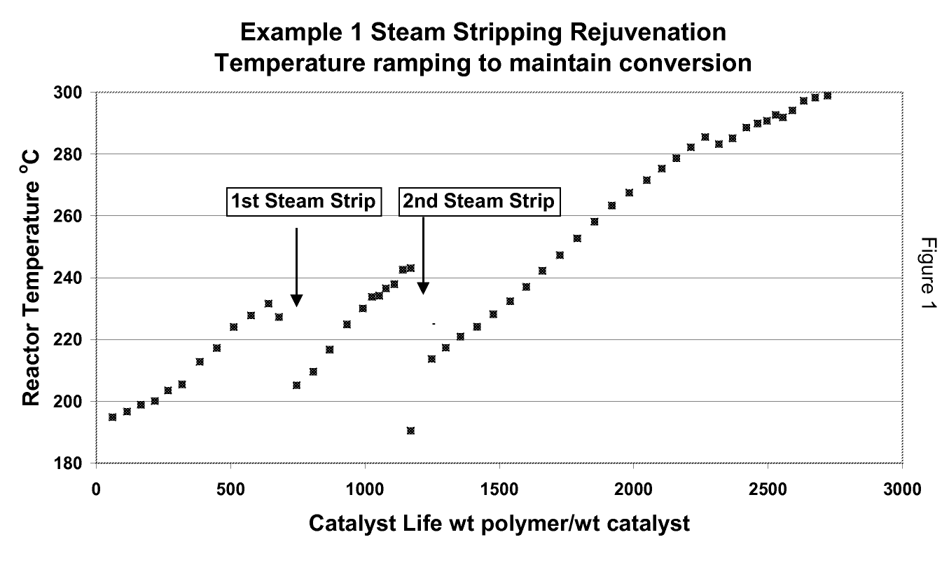

### CROSS REFERENCE TO RELATED PATENT APPLICATIONS

This application is a national stage filing of International Patent Cooperation Treaty Application No. PCT/EP2006/005074 filed May 26, 2006, which claims priority from Great Britain Application 0510887.3 filed on May 31, 2005, the disclosure of which is fully incorporated herein by reference.

### FIELD OF THE INVENTION

This invention relates to liquid phase or supercritical or dense phase olefin oligomerization using molecular sieve catalysts, and especially to catalyst treatment. More especially, it is concerned with rejuvenation of used catalysts from such processes, in particular of crystalline molecular sieve catalysts.

### BACKGROUND OF THE INVENTION

Lower molecular weight organic molecules are converted to higher molecular weight materials by oligomerization. Among such oligomerizations there may be mentioned the conversion of lower olefins, e.g. alkenes, to higher olefins, for example the oligomerization of C₂ to C₆, especially C₃ to C₄, alkenes to olefins in the C₆ to C₁₂ range, and occasionally higher. The oligomers produced are valuable raw materials as feedstocks for further reactions. For example, higher alkenes are converted by the oxo process to aldehydes, which in turn may be converted to acids or alcohols, and esterified to form plasticizers.

The oligomerization may be carried out in the presence of a catalyst, of which many types have been proposed. Among these catalysts are crystalline molecular sieves, e.g. the aluminosilicates, or true zeolites, and the related silicoaluminophosphates (SAPO's) and aluminophosphates (AlPO's).

Examples of olefin oligomerizations carried out in the presence of molecular sieve catalysts are disclosed in EP-A-625 132 and EP-A-746 538.

It is known that the useful life of a molecular sieve catalyst is limited. In the case of a catalyst used in olefin oligomerization, for example, its active sites may be poisoned by contaminants in the feedstock or the sites may become blocked by the build-up of unwanted by-products of reaction.

As examples of contaminants in olefin feedstocks there may be mentioned oxygen-, nitrogen-, and sulphur-containing compounds. It has been found that certain upstream processes in the petrochemical industry form nitriles that are deleterious to catalyst activity. It has recently been discovered that certain sulphur-containing compounds are more deleterious than others, especially those with high desorption temperatures.

In molecular sieve-catalysed olefin oligomerization processes, it has been found that carbonaceous deposits, usually of a higher molecular weight and often referred to as “coke”, block not only the active surface sites on the surface of the molecular sieve, but also the pores of the catalyst, preventing access of reactants to active internal sites as well.

A spent catalyst may be discarded, but disposal may be economically or environmentally unacceptable. A catalyst may be regenerated, by which term is meant the restoration of the activity of the catalyst to or very near to its original activity. Many regeneration methods, however, require high temperatures involving the removal of the catalyst from the reactor, often to a remote location, and lengthy reactor downtime and often substantial expense may be involved. An alternative is catalyst rejuvenation, by which term is meant increasing the activity of a deactivated (a term used to include partially deactivated) catalyst, but not necessarily to its original activity. Rejuvenation methods may be carried out more easily than regeneration, resulting in decreased reactor downtime, in some instances in situ.

In U.S. Pat. No. 4,560,536, there is disclosed a process for oligomerizing ethylene and other olefins to gasoline and distillate range materials over a molecular sieve catalyst of the ZSM-5 type, and regenerating spent catalyst by burning off coke. Similar oxidative regeneration is described in U.S. Pat. No. 5,019,357.

U.S. Pat. No. 6,525,234 describes a process for alkylation of aromatic hydrocarbons by olefins over molecular sieve catalysts. The catalyst is said to be deactivated by materials strongly sorbed under liquid phase alkylation conditions, with low molecular weight nitrogen, oxygen and sulfur compounds being mentioned as poisons. Deactivated catalyst may be reactivated by contact with polar compounds, either in situ, in some cases even while continuing the alkylation reaction, or off-line. The polar compounds mentioned include, inter alia, hydrogen sulfide, dimethyl sulfide, and, among preferred materials, acetic, carbonic, nitric and sulfuric acids, and water, although a preference is also indicated for non-aqueous compounds.

U.S. Pat. No. 5,059,738 describes the reactivation of a catalyst in a process converting methanol to gasoline between about 300° C. and 400° C. in contact with a stream of inert purge gas. The inert gas may include nitrogen, light paraffinic hydrocarbons, and Group VIII gases of the Periodic Table of the Elements. The methanol to hydrocarbon conversion processes, such as the methanol-to-olefins (MTO) process and the methanol-to-gasoline (MTG) process, are known to occur via alkylation and dealkylation reaction steps involving aromatic intermediates. The “coke” formed in these processes therefore contain significant amounts of single up to 4 or 5 multiring aromatics. When the process uses a large pore open structure molecular sieve as catalyst, such as ZSM-5, single ring aromatics are sufficiently small to escape from the catalyst and appear in the product.

U.S. Pat. No. 4,417,086 describes a continuous fluidized bed oligomerization process using a fluidized bed of catalyst in a reaction zone with a first part wherein the olefin feed is introduced and at least partly oligomerized, and a second part where a stripping gas is introduced and at least a part of the olefin oligomers are stripped from the catalyst. The catalyst is circulated between the two parts of the reaction zone. Steam or water vapor may be used as the stripping gas. A further improvement is disclosed whereby the flow of feed to the reaction zone is periodically stopped while continuing to strip the catalyst with stripping gas. The oligomerization feed needs to contain gaseous olefins, and the oligomerization is operated with the olefin feedstock in the gas phase. The activity in such a gas phase oligomerization is significantly lower than with the oligomerization processes where the olefin feedstock is either partially or entirely in the liquid phase, or in the supercritical condition. The gas phase process therefore typically operates at a higher temperature as compared with these other processes, typically above 300° C., where side reactions become significant, such as cracking, olefin disproportionation, hydrogen transfer and dehydrocyclization. These side reactions cause the formation of byproducts such as paraffins, polyunsaturates, aromatics and olefins of other carbon numbers than the true oligomers of the feedstock olefins. These byproducts are acceptable, or even desirable, in certain product uses such as in transportation fuels, but they represent an undesired selectivity loss, and often an unacceptable product contamination, when the oligomer products are intended for the production of chemical derivatives such as alkylates or oxo-alcohols for plasticizers or detergents. In the gas phase oligomerization process of U.S. Pat. No. 4,417,086, the oligomers formed do not readily come off the catalyst, and they therefore are particularly prone to participate in these side reactions. Some of the byproducts, such as the aromatics, are intermediates for the formation of a particular kind of “coke”, containing single and multiring aromatics. That aromatic-containing “coke” is hard to remove from molecular sieve catalysts, and when such deactivated catalysts are rejuvenated, temperatures of above 300° C. are required. There is even no evidence in U.S. Pat. No. 4,417,086 that the rejuvenation at 316° C. is effective in removing also the polynuclear aromatic coke present on the catalyst or trapped in the catalyst pores. Since the typical operating conditions of the gas phase oligomerization process are in the same range, also above 300° C., the equipment complies with the design requirement suitable for this temperature range and the necessary auxiliary equipment is in place and adequate to reach those temperatures. The rejuvenation with inert gas or water vapour above 300° C. therefore does not create an additional burden or complexity on a gas phase oligomerization process.

Oligomerization processes using molecular sieve catalysts at conditions wherein the feedstock is partially or entirely in the liquid phase or in the supercritical or dense phase condition typically operate at temperatures of 300° C. and below. This suppresses side reactions such that higher selectivities to desired true oligomers can be achieved, and the products are of high purity, suitable for the production of chemical derivatives such as alkylates or oxo-alcohols for plasticizers or detergents. Equally important, the carbonaceous deposits formed under these conditions have been found to be predominantly non-aromatic, and to have a hydrogen to carbon atom ratio of between 1.6 and 2.0. If the higher temperature rejuvenation processes known from the gas phase oligomerization process, i.e. above 300° C., are to be applied, additional requirements are put on the equipment designs and on the auxiliary equipment that are not needed for the oligomerization process itself.

There therefore remains a need for a rejuvenation method, applicable to molecular sieve catalysts aged, i.e. deactivated, by use in an olefin oligomerization process under conditions whereby the feedstock is in the liquid phase or in the supercritical or dense phase condition, that does not bring with it the additional requirements on the equipment designs nor the need for auxiliary equipment that is not needed for the oligomerization process itself.

We have now found that the high molecular weight carbonaceous deposits in the oligomerization processes wherein the feedstock is in the liquid phase or in the super-critical condition, is different and of a softer, non-aromatic nature and that the molecular sieve catalysts deactivated by use in such processes can be rejuvenated at milder conditions at or below 300° C. This means that the need for more stringent equipment design criteria and for extra auxiliary equipment can be avoided.

There remains a need for a method in which a molecular sieve catalyst deactivated by use in oligomerizing olefins under conditions wherein the feedstock is in the liquid phase or in the supercritical condition may be readily rejuvenated, advantageously if desired in situ. There also remains a need for a method that rejuvenates a molecular sieve catalyst that has been deactivated by a feedstock contaminated with sulphur and/or nitrogen compounds.

### SUMMARY OF THE INVENTION

The present invention provides a method of rejuvenating a molecular sieve catalyst deactivated by use in olefin oligomerization under conditions wherein the feedstock is in the liquid phase or in the supercritical condition, which method comprises contacting the deactivated catalyst with steam at a temperature of at most 300° C. for a time sufficient to effect an increase in catalytic activity of the deactivated catalyst.

The invention also provides a process for olefin oligomerization under conditions wherein the feedstock is in the liquid phase or in the supercritical condition, which comprises contacting an olefinic feedstock with a molecular sieve catalyst under oligomerization conditions wherein the feedstock is in the liquid phase or in the supercritical condition for a period which results in a deactivation of the catalyst, carrying out the rejuvenation method of the invention on the deactivated catalyst, and contacting an olefinic feedstock under oligomerization conditions wherein the feedstock is in the liquid phase or in the supercritical condition with the rejuvenated catalyst, optionally repeating rejuvenation and oligomerization one or more times.

### DETAILED DESCRIPTION OF THE INVENTION

The terms “supercritical” and “dense” as related to a fluid phase or conditions are terms that are herein used interchangeably. Both refer to a fluid at a temperature and a pressure above its thermodynamic critical point. The pressure-temperature phase diagram for a pure substance typically shows the conditions where liquid and vapor may coexist as a line ending in a maximum at what is defined as the thermodynamic critical point. The same diagram looks different for a mixture of compounds that have different boiling points. When for such a mixture, the initial boiling point temperatures and the initial dew point temperatures for the same pressures are traced, so as to envelop the two-phase region where vapor and liquid may coexist, typically a tear- or bell-shaped curve is obtained. The thermodynamic critical point is then defined as where the two-phase envelope reaches a maximum in pressure. The critical pressure is thus defined as the pressure above which no two-phases may coexist at any temperature. The critical temperature is then defined as the temperature at which the two-phase envelope reaches that maximum pressure.

As molecular sieves to be rejuvenated, there may be mentioned silicoaluminates, or true zeolites, for example, ZSM-5, ZSM-11, ZSM-12, ZSM-21, ZSM-22, ZSM-23, ZSM-35, ZSM-38, ZSM-45, ZSM-48, MCM-22, MCM-49 and zeolites β, A, X and Y. Apart from zeolites proper, there may be mentioned the silicoaluminophosphates and the aluminophosphates (SAPO's and ALPO's) especially SAPO-18, 34, 35, 44 and 47 and ALPO-5, 11, 18, 31, 34, 36, 37 and 46 and the metal-containing forms thereof.

The catalyst may be in any form, especially those typically used in hydrocarbon conversions, for example as a powder or extrudate.

Olefin oligomerization is normally carried out at superatmospheric pressure and elevated temperature, so the feedstock, comprising reactant olefins and any diluents, is in the liquid phase or in the super-critical condition. The catalyst is typically placed in a fixed bed, and this may be inside a tubular or a chamber reactor. When catalyst rejuvenation according to the invention is to be carried out in situ, supply of feedstock to the reactor is interrupted, and reactor pressure advantageously lowered, for example to a pressure of at most about 2 or 3 atmospheres (references in this specification to pressure are to absolute pressure except where indicated), 2 or 3 bar, 200 or 300 kPa. Advantageously, however, the pressure is reduced to sub-atmospheric, more advantageously to at most 50 kPa, preferably at most 30 kPa and most preferably at most 20 kPa, for rejuvenation.

Typical oligomerization temperatures in processes wherein the feedstock is in the liquid phase or in the supercritical condition are in the range of from 150° C. to 300° C., more especially 150° C. to 250° C., and preferably from 200° C. to 250° C. Rejuvenation is advantageously carried out at a temperature in these ranges, although temperatures up to 350° C., for example from 150° C. to 300° C., or from 155° C. up to below 300° C., more preferably from 200° C. to 250° C., may be used, whether in situ or elsewhere. Conveniently, if rejuvenation is effected in situ, it is carried out at a temperature corresponding to that obtaining in the reactor immediately beforehand. Advantageously, the pressure and temperature are such that the steam is superheated.

Rejuvenation is advantageously carried out over a period of, for example, from 1 to 12 hours, preferably for from 2 to 4 hours. The steam flow rate is advantageously at a volume hourly space velocity (VHSV) in the range of from 0.2 to 1 hr⁻¹.

The rejuvenation method should be carried out in the substantial absence of substances that deleteriously affect rejuvenation. For example, the steam supply is advantageously substantially free from sulfur compounds, in which context is required that the supply contains less than 1 ppm by weight sulfur compounds measured as sulfur.

As the activity of a catalyst is reduced (a result of build-up of high molecular weight reaction products (coke), poisoning of active sites by feedstock contaminants, or usually both), it is common practice to maintain conversion rate, measured in a continuous process by the percentage of active feedstock reactants converted to product, as nearly constant as possible by increasing the reaction temperature. The maximum temperature is limited by a number of factors, including reactor design, especially the practicality of water-cooling, and the increase in coke formation at higher temperatures.

Concomitantly, the extent of rejuvenation, i.e. the increase in catalytic activity, is observable by a reduction in the temperature required for the catalysed reaction to proceed at a given conversion rate, with other conditions, e.g., reactant flow rate, being kept constant. Advantageously, the required reaction temperature after rejuvenation is at least 5 degrees C., preferably at least 10 degrees C., and more preferably at least 25 degrees C. below that required before rejuvenation.

It has been found that rejuvenation is the more effective the less the extent to which deactivation has been allowed to proceed, and the invention accordingly also provides a process for olefin oligomerization over a molecular sieve catalyst under conditions wherein the feedstock is in the liquid phase or in the supercritical condition, which comprises interrupting the reaction after the temperature required to carry out the reaction under otherwise constant conditions has risen by at most 50 degrees C., advantageously at most 40 degrees C., and preferably at most 30 degrees C., and rejuvenating the deactivated catalyst by contact with steam to increase its catalytic activity, optionally repeating the cycle of reaction and rejuvenation one or more times.

Treatment of deactivated catalyst by steam is very effective and accordingly may be the sole treatment of the catalyst before being returned to service. However, it is also within the scope of the invention to use the method of the invention prior to taking the catalyst offline for regeneration by other methods, for example, those known in the art.

The following example, when read in conjunction with the accompanying drawing, a graph which shows the increase in reactor temperature with time to maintain constant conversion rate together with two steam treatments, illustrates the invention. Parts and percentages are by weight unless otherwise indicated.

To the knowledge of the inventors, steam stripping for rejuvenation of deactivated molecular sieve catalysts has been tried in many processes, including clay treating and ethylbenzene or cumene production by alkylation, typically immediately before the catalyst was to be regenerated. To date, the activity improvement was found to be insignificant or at least too small to bring any economic benefit. This example for the first time demonstrates on a commercial scale that steam rejuvenation of a deactivated molecular sieve catalyst works and that the technical benefits brought by it may be sufficient to be of economic importance.

### EXAMPLE

A feedstock of 65% butenes/35% butanes by weight was saturated with water vapor by passing it through a vessel containing water at 39° C. The feed was preheated and then passed downward through a tubular reactor containing H-ZSM-22 catalyst at a pressure of 79 barg (80 bar, 8.0 MPa absolute) at a constant weight hourly space velocity (WHSV) of 7 hr⁻¹ and an initial temperature of 195° C., the temperature being subsequently raised to maintain the initial conversion rate. As can be seen from the graph, over a period in which about 600 g of oligomer per g of catalyst had been produced, the reactor temperature had to be increased to 232° C. to maintain the conversion rate. The feed was interrupted, and steam at a temperature of 230° C. and a pressure of 0.5 bar absolute was passed through the reactor at a VHSV of 2 hr⁻¹ for 6 hours. On resuming the olefin feed, it was found that the temperature necessary to maintain the previous conversion rate had fallen to 205° C. During a period in which a further 600 g of oligomer per g of catalyst was produced, the reactor temperature had to be increased to 242° C. to maintain the conversion rate. A second steam treatment, under the same conditions as the first, enabled the reactor temperature to be reduced to 215° C. for the same conversion rate.

What we claim:

1. A process for rejuvenating a molecular sieve catalyst placed in a fixed bed deactivated by use in olefin oligomerization under oligomerization conditions wherein the feedstock is in the liquid phase or in the supercritical condition, which method comprises contacting the deactivated catalyst with steam at a temperature of at most 300° C. for at least 1 hour to effect an increase in catalytic activity of the deactivated catalyst.

2. The process as claimed in claim 1, wherein rejuvenation is carried out at a pressure of at most 100 kPa.

3. The process as claimed in claim 2, wherein rejuvenation is carried out at a pressure of at most 30 kPa.

4. The process as claimed in claim 3, wherein rejuvenation is carried out at a pressure of at most 20 kPa.

5. The process as claimed in claim 1, wherein rejuvenation is carried out at a temperature in the range of from 150° C. to 300° C.

6. The process as claimed in claim 5, wherein rejuvenation is carried out at a temperature within the range of from 200° C. to 250° C.

7. The process as claimed in claim 1, wherein rejuvenation is carried out over a period of from 2 to 4 hours.

8. The process as claimed in claim 1, wherein rejuvenation is carried out at a steam flow rate (VHSV) of from 0.2 to 1 hr⁻¹.

9. The process as claimed in claim 1 wherein the molecular sieve comprises ZSM-22, ZSM-57 or MCM-22.

10. A process for the oligomerization of an olefinic feedstock under conditions wherein the feedstock is in the liquid phase or in the supercritical condition, which comprises contacting the feedstock under such oligomerization conditions with a molecular sieve catalyst that has been rejuvenated by the process according to claim 1.

11. The process according to claim 10 which comprises contacting the olefinic feedstock with the molecular sieve catalyst for a period which results in a deactivation of the catalyst before carrying out the rejuvenation process, optionally repeating rejuvenation and oligomerization one or more times.

12. The process according to claim 11 which comprises interrupting the oligomerization reaction after the temperature required to carry out the reaction under otherwise constant conditions has risen by at most 50 degrees C. before carrying out the rejuvenation process.
